(19) United States
(12) Patent Application Publication
(10) Pub. No.: US 2002/0095109 A1
(43) Pub. Date: Jul. 18, 2002

(54) Safety drop infusion/transfusion set capable of preventing recovered hypodermic needle from being pushed out

(76) Inventor: Fu-Nan Chen, Sangchung City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/955,986

(22) Filed: Sep. 20, 2001

(30) Foreign Application Priority Data
Jan. 18, 2001 (TW) 90200987

(51) Int. Cl. A61M 37/00

(52) U.S. Cl. 604/6.16

(57) ABSTRACT

Safety drop infusion/transfusion set capable of preventing recovered hypodermic needle from being pushed out, including: a sleeve slidably fitted on an infusion/transfusion catheter, a locating flange being formed on inner face of the wall of bottom end of the sleeve; and a needle holder formed with an axial tunnel. A top section of the needle holder is formed as a seat body for fitting with a hypodermic needle. A bottom section of the needle holder is formed as a connecting section for tightly fitting with the infusion/transfusion catheter. When downward pulling the infusion/transfusion catheter, the needle holder is drivingly retracted and latched with the locating flange of the sleeve. The hypodermic needle along with the needle holder is retracted and hidden in the sleeve. Therefore, the hypodermic needle recovered in the sleeve is prevented from being again pushed out of the sleeve.

### BACKGROUND OF THE INVENTION

1. Field of the Invention

The present invention is related to a safety drop infusion/transfusion set capable of preventing recovered hypodermic needle from being pushed out.

2. Description of the Prior Art

The existing infusion or transfusion needle includes an injection needle and an infusion/transfusion catheter connected with the injection needle. After the injection is completed, a needle sheath is fitted onto the injection needle and then the injection needle is discarded. The needle sheath has a very small fitting hole so that it often takes place that a medical man is incautiously impaled when inserting the needle into the needle sheath. As a result, the medical man may get infected with diseases such as AIDS, hepatitis, etc. Moreover, when treating medical wastes, an operator may get impaled in case the needle sheath slips from the needle.

U.S. Pat. No. 5,931,815 discloses a retractable hidden-type multifunctional safety infusion dropper which includes a connecting tube 1 and a wing-type sheath 4 slidably fitted around the connecting tube 1. The outer edge of the connecting tube 1 is formed with an upper latch section 11 and a lower latch section 12. The bottom end of the connecting tube 1 is connected with a liquid medicine infusion catheter 2. The top end thereof is connected with an injection needle 3. The inner edge of the fitting hole 41 of the wing-type sheath 4 is formed with a connecting section 411 for up and down slidably latching with the upper and lower latch sections 11, 12 of the connecting tube 1. When the connecting section 411 of the sheath 4 is firmly latched with the upper latch section 11, the injection needle 3 is totally retracted and hidden in the fitting hole 41 of the sheath 4 without protruding outside so as to avoid impalement of other people.

In the above structure, the connecting tube 1 is an elongated rod made of plastic material. When the connecting section 411 of the sheath 4 is firmly latched with the upper latch section 11 of the connecting tube 1, the injection needle 3 is totally hidden in the fitting hole 41 of the sheath 4. However, the bottom section of the connecting tube 1 lengthwise protrudes out of the sheath 4. The connecting tube 1 is a hard article so that when recovered, the connecting tube 1 may serve as a force application arm and slide relative to the sheath 4. Accordingly, the connecting tube 1 may be latched from the sheath 4 and the injection needle 3 may be again pushed outward and exposed to outer side. This leads to danger in recovering and treating the medical wastes.

Moreover, only the needle holder 31 at bottom end of the injection needle 3 is fitted around the connecting section 14 of the connecting tube 1 without being fixedly locked thereon. Therefore, after the injection is completed and when a user tries to fit the wing-type sheath 4 onto the injection needle 3, the front end of the sheath 4 is extremely likely to touch and outward push the needle holder 31 away from the connecting section 14. As a result, the injection needle 3 may be thrown out to hurt other people.

### SUMMARY OF THE INVENTION

It is therefore a primary object of the present invention to provide a safety drop infusion/transfusion set capable of preventing recovered hypodermic needle from being pushed out, including: a sleeve slidably fitted on an infusion/transfusion catheter, a locating flange being formed on inner face of the wall of bottom end of the sleeve; and a needle holder formed with an axial tunnel. A top section of the needle holder is formed as a seat body for fitting with a hypodermic needle. A bottom section of the needle holder is formed as a connecting section for tightly fitting with the infusion/transfusion catheter. When downward pulling the infusion/transfusion catheter, the needle holder is drivingly retracted and latched with the locating flange of the sleeve. The hypodermic needle along with the needle holder is retracted and hidden in the sleeve. When an external force is exerted to reversely push the infusion/transfusion catheter, the infusion/transfusion catheter is flexed and deformed so that it is impossible to push the needle holder out of the sleeve. Therefore, the hypodermic needle recovered in the sleeve is prevented from being again pushed out of the sleeve.

The present invention can be best understood through the following description and accompanying drawings wherein:

### BRIEF DESCRIPTION OF THE DRAWINGS

FIG. 1 is a perspective exploded view of the present invention;

FIG. 2 is a perspective assembled view of the infusion/transfusion set of the present invention;

FIG. 3 is a sectional view taken along line 3-3 of FIG. 2;

FIG. 4 shows the present invention in a state of injection;

FIG. 5 shows that the hypodermic needle is retracted into the sleeve of the present invention; and

FIG. 6 is an enlarged view of a part of FIG. 5.

### DETAILED DESCRIPTION OF THE PREFERRED EMBODIMENTS

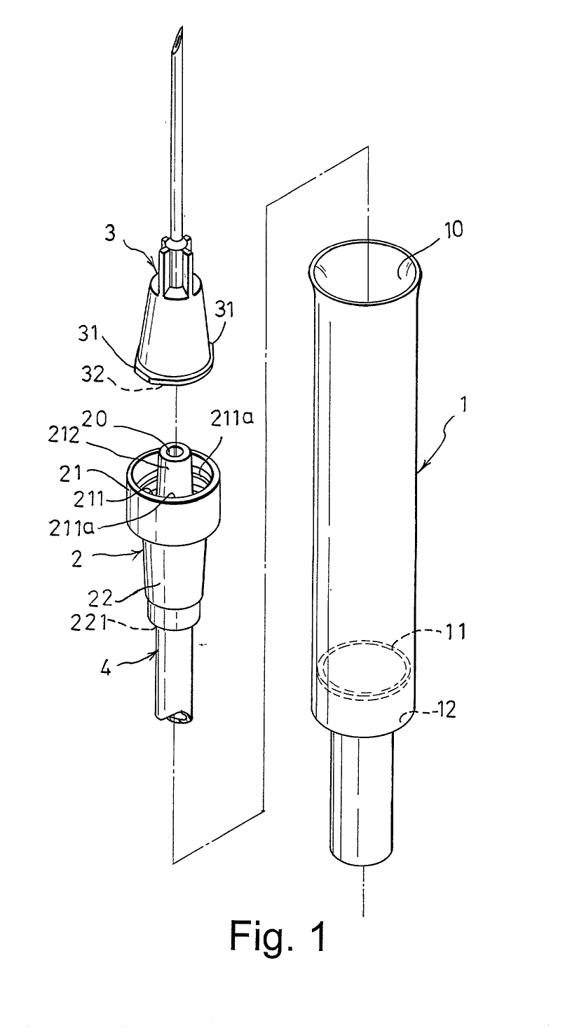

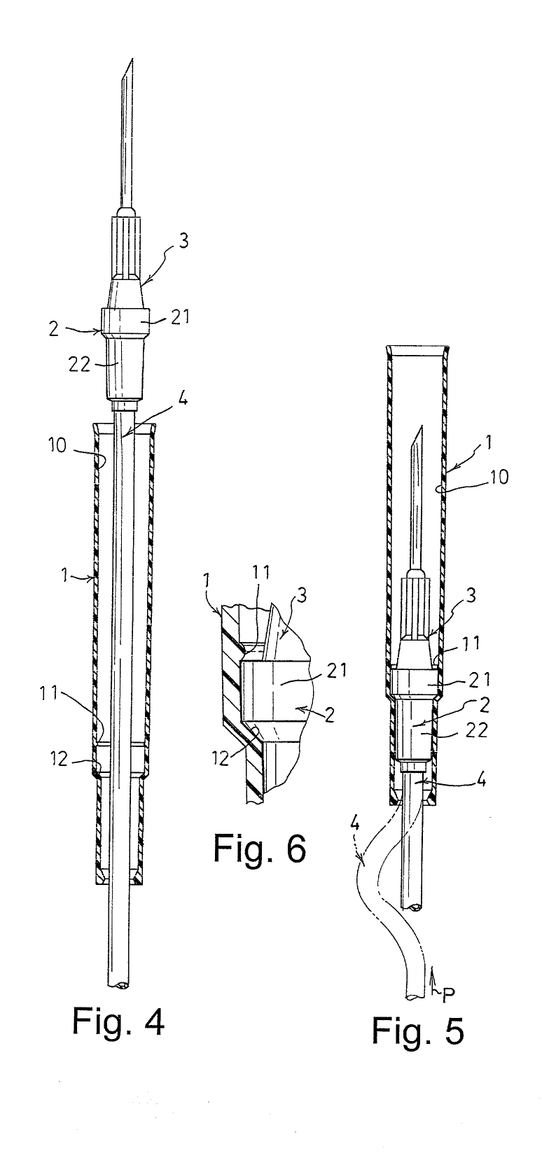

Please refer to FIGS. 1 to 6. The safety drop infusion/transfusion set of the present invention includes: a sleeve 1 slidably fitted on an infusion/transfusion catheter 4, a locating flange 11 being formed on inner face of the wall of bottom end of the sleeve 1; and a needle holder 2 formed with an axial tunnel 20. The top section of the needle holder 2 is formed as a seat body 21 for fitting with a hypodermic needle 3. The bottom section of the needle holder 2 is formed as a connecting section 22 for tightly fitting with the infusion/transfusion catheter 4. When downward pulling the infusion/transfusion catheter 4, the needle holder 2 is drivingly retracted and latched with the locating flange 11 of bottom end of the sleeve 1. Under such circumstance, the hypodermic needle 3 along with the needle holder 2 is retracted and hidden in the sleeve 1. When an external force is exerted to reversely push the infusion/transfusion catheter 4, the infusion/transfusion catheter 4 is curved and deformed so that it is impossible to push the needle holder 2 out of the sleeve 1. Therefore, the hypodermic needle 3 is prevented from being pushed out of the sleeve 1 and is safely recovered in the sleeve 1.

Referring to FIGS. 1, 4, 5 and 6, the sleeve 1 is formed with a central axial passage 10 through which the infusion/transfusion catheter 4 is passed. The locating flange 11 is formed on inner face of the wall of the passage 10 near bottom end thereof. A stop shoulder 12 is defined below the locating flange 11 for stopping the needle holder 2 from downward slipping out.

Referring to FIGS. 1, 2 and 3, the needle holder 2 is made of plastic material by integral injection molding. The needle holder 2 is formed with a central axial tunnel 20 through which a liquid medicine or blood flows. The seat body 21 is formed on top section of the needle holder 2. The end face of the seat body 21 is formed with an annular fitting socket 21 1. The circumference of the fitting socket 211 is formed with double inner threads 211a for screwing with double flanges 31 of bottom end of the hypodermic needle 3. A fitting post 212 axially projects from the center of the seat body 21 for snugly fitting with a fitting hole 32 of the bottom end of the hypodermic needle 3. The connecting section 22 is formed on the bottom section of the needle holder 2. The bottom end of the connecting section 22 is formed with a connecting dent 221 in which the head end of the infusion/transfusion catheter 4 is tightly connected.

The infusion/transfusion catheter 4 and the periphery of the connecting dent 221 can be connected with each other by high frequency wave, whereby the infusion/transfusion catheter 4 is tightly connected with the bottom end of the needle holder 2.

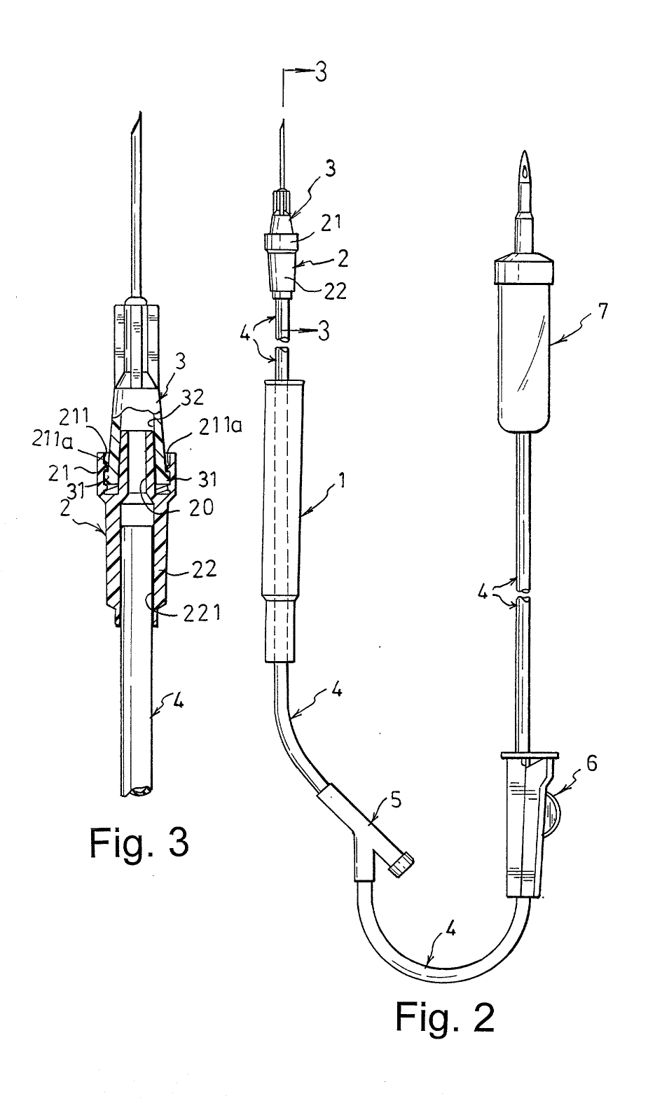

As shown in FIG. 2, the infusion/transfusion catheter 4 can be equipped with a Y-shaped connecting tube 5, a flow controller 6, a drop bottle plug 7, etc. for drop infusion or transfusion. However, the injection measure of the present invention is not limited. The present invention is also applicable to other hypodermic needles such as scalp needle and intravenous injection needle.

Referring to FIGS. 1 and 2, in use of the present invention, the needle holder 2 can be slided out of and separated from the sleeve 1. At this time, a medical man can turn and fit the hypodermic needle 3 onto the seat body 21 of the needle holder 2 to complete preparation of injection.

After the injection is completed, the medical man can hold the sleeve 1 with one hand and downward pull the infusion/transfusion catheter 4 at the bottom end of the sleeve 1 with the other hand as shown in FIGS. 5 and 6. At this time, the needle holder 2 is drivingly slided into the passage 10 of the sleeve 1 that is, the hypodermic needle 3 is drivingly retracted and hidden in the sleeve 1. The infusion/transfusion catheter 4 is further downward pulled until the top edge of the seat body 21 of the needle holder 2 is latched under the locating flange 11 of the sleeve 1. At this time, the needle holder 2 is firmly latched in the sleeve 1 and prevented from upward slipping out of the sleeve 1. Accordingly, the hypodermic needle is prevented from being exposed to outer side so as to avoid impalement of medical personnel. The double flanges 31 of bottom end of the hypodermic needle 3 are locked with the double inner threads 211a of the fitting dent 211 of the seat body 21. Therefore, when pulling the needle holder 2 into the sleeve 1, the hypodermic needle 3 screwed in fitting dent 211 of the seat body 21 will not be pushed away by the sleeve 1. In addition, the front edge of the passage 10 of the sleeve 1 is trumpet-shaped so that the needle is guided and smoothly retracted to ensure safety.

Referring to FIG. 5, after the needle holder 2 and the hypodermic needle 3 are totally hidden in the sleeve 1, only the infusion/transfusion catheter 4 protrudes from the bottom end of the sleeve 1. Even if an external force P is exerted onto the infusion/transfusion catheter 4 to upward push the needle holder 2 latched in the bottom end of the sleeve 1, the infusion/transfusion catheter 4 will be flexed as shown by phantom line of FIG. 5. Therefore, it is impossible for the external force P to push the needle holder 2 and unlatch the needle holder 2 from the sleeve 1. Accordingly, once the needle holder 2 is latched in the sleeve 1, the needle holder 2 as well as the hypodermic needle 3 are firmly located and hidden in the sleeve 1 without protruding outward. Therefore, the hypodermic needle 3 can be safely recovered and processed.

According to the above arrangement, the present invention has the following advantages:

1. After used, the hypodermic needle 3 are truly located and hidden the sleeve 1 without protruding outward so as to avoid impalement of medical personnel and infection of diseases.

2. After the needle holder 2 is latched in the sleeve 1, it is impossible for external force to push the needle holder and unlatch the needle holder from the sleeve 1. Accordingly, the hypodermic needle 3 is firmly located and hidden in the sleeve 1 without protruding outward. Therefore, the medical waste can be safely recovered and processed.

3. The double flanges 31 of bottom end of the hypodermic needle 3 are locked with the double inner threads 21 la of the fitting dent 211 of the seat body 21. Therefore, when pulling the needle holder 2 into the sleeve 1, the hypodermic needle 3 screwed in fitting dent 211 of the seat body 21 will not be pushed away by the sleeve. In addition, the front edge of the passage 10 of the sleeve 1 is trumpet-shaped so that the needle is guided and smoothly retracted to ensure safety.

The above embodiments are only used to illustrate the present invention, not intended to limit the scope thereof. Many modifications of the above embodiments can be made without departing from the spirit of the present invention.

What is claimed is:

1. Safety drop infusion/transfusion set capable of preventing recovered hypodermic needle from being pushed out, comprising:
a sleeve slidably fitted on an infusion/transfusion catheter, a locating flange being formed on inner face of the wall of bottom end of the sleeve; and
a needle holder formed with an axial tunnel, a top section of the needle holder being formed as a seat body for fitting with a hypodermic needle, a bottom section of the needle holder being formed as a connecting section for tightly fitting with the infusion/transfusion catheter, whereby when downward pulling the infusion/transfusion catheter, the needle holder is drivingly retracted and latched under the locating flange of bottom end of the sleeve and the hypodermic needle along with the needle holder is totally retracted and hidden in the sleeve without protruding outward, when an external force is exerted to reversely push the infusion/transfusion catheter, the infusion/transfusion catheter being flexed and deformed so that it is impossible to push and unlatch the needle holder from the sleeve, whereby the hypodermic needle recovered in the sleeve is prevented from being again pushed out of the sleeve.

2. Safety drop infusion/transfusion set capable of preventing recovered hypodermic needle from being pushed out as claimed in claim 1, wherein the sleeve is formed with a central axial passage through which the infusion/transfusion catheter is passed, the locating flange being formed on inner face of the wall of the passage near bottom end thereof, a stop shoulder being defined below the locating flange for stopping the needle holder from downward slipping out.

3. Safety drop infusion/transfusion set capable of preventing recovered hypodermic needle from being pushed out as claimed in claim 1, wherein the needle holder is made of plastic material by integral injection molding, the needle holder being formed with a central axial tunnel through which a liquid medicine or blood flows, the seat body being formed on top section of the needle holder, the end face of the seat body being formed with an annular fitting socket, a circumference of the fitting socket being formed with double inner threads for screwing with double flanges of bottom end of the hypodermic needle, a fitting post axially projecting from the center of the seat body for snugly fitting with a fitting hole of the bottom end of the hypodermic needle, the connecting section being formed on the bottom section of the needle holder, the bottom end of the connecting section being formed with a connecting dent in which the head end of the infusion/transfusion catheter is tightly connected.

4. Safety drop infusion/transfusion set capable of preventing recovered hypodermic needle from being pushed out as claimed in claim 3, wherein the infusion/transfusion catheter and the periphery of the connecting dent of the connecting section are connected with each other by high frequency wave, whereby the infusion/transfusion catheter is tightly connected with the bottom end of the needle holder.
